(19) United States
(12) Patent Application Publication
(10) Pub. No.: US 2025/0248954 A1
(43) Pub. Date: Aug. 7, 2025

(54) PHARMACEUTICAL COMPOSITION FOR PREVENTING OR TREATING DIABETIC STROKE, CONTAINING RAGE ANTAGONIST AND AGE SCAVENGER

(71) Applicant: AJOU UNIVERSITY INDUSTRY-ACADEMIC COOPERATION FOUNDATION, Gyeonggi-do (KR)

(72) Inventors: Jin Soo LEE, Seoul (KR); Bok Seon YOON, Gyeonggi-do (KR)

(73) Assignee: AJOU UNIVERSITY INDUSTRY-ACADEMIC COOPERATION FOUNDATION, Gyeonggi-do (KR)

(21) Appl. No.: 18/855,162

(22) PCT Filed: Mar. 14, 2023
(86) PCT No.: PCT/KR2023/003398

§ 371 Date: Oct. 8, 2024

(30) Foreign Application Priority Data
Apr. 15, 2022 (KR) 10-2022-0047059

(51) Int. Cl. A61K 31/166; A61K 31/155; A61P 3/10; A61P 9/10

(52) U.S. Cl. CPC A61K 31/166; A61K 31/155; A61P 3/10; A61P 9/10

(57) ABSTRACT

The present invention relates to a pharmaceutical composition for preventing or treating diabetic stroke, containing at least one of a RAGE (receptor for advanced glycation end-products) antagonist or an AGE (advanced glycation end-products) scavenger. Administration of the composition to a subject can prevent or treat stroke with hyperglycemia. More specifically, administration of the composition to a subject inhibits brain injury caused by stroke with hyperglycemia.

### TECHNICAL FIELD

The present invention relates to a pharmaceutical composition for preventing or treating diabetic stroke, containing a RAGE antagonist and an AGE scavenger.

### BACKGROUND ART

According to statistics on diseases of national interest published by the Health Insurance Review and Evaluation Service, the number of patients receiving treatment for stroke increased by about 13% from 538, 443 in 2015 to 613,824 in 2019. In addition, according to the statistics on causes of death in Korea announced by the Statistics Korea in 2020, cerebrovascular disease ranked fourth among the causes of death in Korea.

Stroke refers to a neurological symptom that occurs when a blood vessel supplying blood to a part of the brain is blocked or burst, resulting in damage to that part of the brain. Stroke is broadly classified into two types.

The first type is the blockage of blood vessels, which damages a part of the brain that was supplied with blood by the blood vessels. This is also called cerebral infarction, ischemic stroke, or infarct stroke. The second type is the rupture of a blood vessel in the brain, which causes blood to pool in the brain and damages that part of the brain. This is called hemorrhage or hemorrhagic stroke.

Since once necrotic brain tissue cannot be restored to its previous state even by any treatment, minimizing damage to brain tissue is most important. When a stroke occurs, brain damage may occur, and language impairment, cognitive impairment, and physical disability may remain as aftereffects due to the brain damage.

The following studies have been continuously reported: a study showed that even in acute ischemic stroke patients without diabetes, 30 to 40% of the patients show hyperglycemia, worsening clinical outcomes (Luitse M J et al., 2012); a study showed that the risk of bleeding is doubled for every 5.5 mM increase in blood glucose level in patients with acute ischemic stroke (Bruno et al., 2002); and a study showed that in the clinical patient group with cerebral ischemia-reperfusion, patients with hyperglycemia upon admission to the hospital show a larger cerebral infarction volume and more frequent cerebral infarction hemorrhage (Seongjun Lee, Jinsu Lee et al., Sci Rep, 2019). From the above studies, it can be seen that the prognosis of acute ischemic stroke is worse in patients with diabetes and hyperglycemia.

However, although various mechanisms have been proposed, there are still no clear causal factors. A study (Johnston et al., 2019) showed that in the SHINE clinical trial, glucose levels were thoroughly controlled immediately after cerebral infarction, but the prognosis of cerebral infarction was not improved. This suggests that there is no treatment for the mechanism of worsening and brain protection.

In addition, there have been continued reports that the worsening of prognosis by diabetes or hyperglycemia appears not only in cerebral infarction but also in intracerebral hemorrhage (ICH) (Saxena A et al. [INTERACT2], 2016) (Song E C et al, 2003), traumatic brain injury (TBI) (Rovlias A, Kotsou S, 2000), subarachnoid hemorrhage (SAH) (Frontera J A et al, 2006), and the like.

### PRIOR ART DOCUMENTS

### Patent Documents

(Patent Document 0001) Korean Patent No. 10-1516352 (May 4, 2015)

### DISCLOSURE

### Technical Problem

One aspect provides a pharmaceutical composition for preventing or treating diabetic stroke, containing at least one of a RAGE (receptor for advanced glycation end-products) antagonist or an AGE (advanced glycation end-products) scavenger.

Another aspect provides a method for preventing or treating diabetic stroke, comprising a step of administering a pharmaceutical composition for preventing or treating diabetic stroke, containing at least one of a RAGE (receptor for advanced glycation end-products) antagonist or an AGE (advanced glycation end-products) scavenger, to a subject likely to develop stroke with hyperglycemia or suffering from stroke with hyperglycemia.

The subject likely to develop stroke with hyperglycemia or suffering from stroke with hyperglycemia may be an animal, including or excluding humans.

### Technical Solution

One aspect provides a pharmaceutical composition for preventing or treating diabetic stroke, containing at least one of a RAGE (receptor for advanced glycation end-products) antagonist or an AGE (advanced glycation end-products) scavenger.

The RAGE is called the receptor for advanced glycation end-products and is a cell membrane multiligand receptor that binds to various types of ligands belonging to the immunoglobulin family. Unlike other multiligand receptors, the various types of ligands that bind thereto are all intracellular products. The advanced glycation end-products (AGEs) result from non-enzymatic glycation and oxidation of proteins, appear in stress-related conditions similar to those in autoimmune connective tissue diseases, and may be formed by oxidation in inflamed tissues or by the myeloperoxidase pathway.

The RAGE antagonist may be a substance that inhibits the action of the RAGE by interfering with the interaction between the RAGE and its ligands.

In one embodiment, the RAGE antagonist may be an AGE antagonist, an HMGB1 antagonist, an S100A4 antagonist, an S100β antagonist, an S100P antagonist, an amyloid-ß (Aß) antagonist, or a RP1 antagonist, without being limited thereto.

In one embodiment, the RAGE antagonist may be FPS-ZM1, HMB1 box A, ethyl pyruvate, or azeliragon, without being limited thereto.

The HMGB-1 (high mobility group box 1), S100A4 (S100 calcium binding protein A4), S100β, S100P (S100 calcium binding protein P), and RP1 (RP1 axonemal microtubule associated) are substances corresponding to ligands for RAGE, and may be substances that promote the action of RAGE by binding to the RAGE. For example, the HMGB-1 antagonist may be HMGB1 box A, or ethyl pyruvate, without being limited thereto.

The RAGE antagonist may be FPS-ZM1. FPS-ZM1 may competitively bind to the domain of RAGE and inhibit the interaction of RAGE with ligands such as AGEs, HMGB1, S100A4, S100β, S100P, amyloid-ß (AB), and RP1, thereby reducing signaling caused by binding to RAGE. The FPS-ZM1 is a C₂₀H₂₂ClNO compound and refers to a compound represented by Formula 1 below.

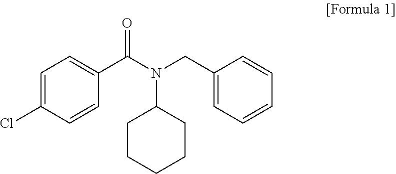 [SMILES: O=C(c1ccc(Cl)cc1)N(Cc1ccccc1)C1CCCCC1]

The RAGE antagonist may be azeliragon. Azeliragon may be referred to as PF-04494700 or TTP488, and refers to a compound represented by Formula 2 below. Azeliragon may be an oral small molecule inhibitor of RAGE.

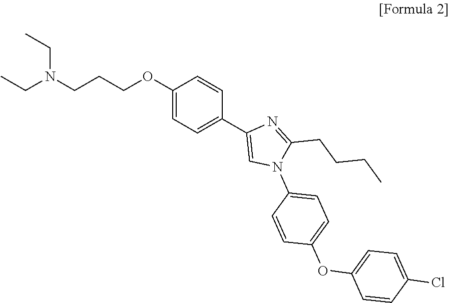 [SMILES: CCCCc1nc(-c2ccc(OCCCN(CC)CC)cc2)cn1-c1ccc(Oc2ccc(Cl)cc2)cc1]

The AGE scavenger may inhibit the formation of AGEs.

In one embodiment, the AGE scavenger may be vitexin, isovitexin, aminoguanidine, thymoquinone, epigallocatechin gallate, rutin, aspirin, or penicillamine.

In one embodiment, the diabetic stroke may be stroke with hyperglycemia. For example, the diabetic stroke may include, but is not limited to, stroke with diabetes, stroke with hyperglycemia, subarachnoid hemorrhage with hyperglycemia, and traumatic brain injury with hyperglycemia.

In one example, diabetic stroke was induced by inducing hyperglycemia in a rat and inducing stroke 34 days later, and hyperglycemic stroke was induced by inducing hyperglycemia in a rat and inducing stroke on day 4. It was confirmed that, in both groups, brain injury was significantly reduced when treated with a RAGE antagonist or an AGE scavenger.

The prevention or preventing refers to any action that inhibits or delays the onset and worsening after onset of diabetic stroke by administering the composition of the present invention.

The treatment or treating refers to any action that alleviates or ameliorates diabetic stroke symptoms and worsening after onset thereof by administering the composition of the present invention.

In one example, it was confirmed that, when stroke occurred in an experimental group with induced hyperglycemia, brain injury in the group significantly increased compared to that in a normoglycemic group. In addition, in one example, it was confirmed that brain injury was significantly reduced when a RAGE antagonist was administered to an experimental group with induced hyperglycemia, and that brain injury was significantly reduced when an AGE scavenger was administered to an experimental group with induced diabetes.

The pharmaceutical composition of the present invention may be used as a single formulation, or it may be prepared and used as a combination formulation further containing a drug known to be effective against stroke with hyperglycemia or against diabetic stroke.

The pharmaceutical composition of the present invention may further contain a pharmaceutically acceptable carrier.

The “pharmaceutically acceptable” means neither significantly stimulating an organism nor inhibiting the biological activity and characteristics of an active material administered.

The carrier may be a natural or non-natural carrier. Depending on the formulation, the composition may be formulated using various carriers such as diluents or excipients, including fillers, extenders, binders, wetting agents, disintegrants, and surfactants. For example, solid formulations for oral administration include tablets, pills, powders, granules, capsules, etc. Such solid formulations are prepared by mixing one or more compounds with at least one excipient, for example, starch, calcium carbonate, sucrose, lactose, gelatin, or the like. In addition to simple excipients, lubricants are also used, such as magnesium stearate, talc, etc. Liquid formulations for oral administration include suspensions, oral solutions, emulsions, and syrups, and may contain, in addition to commonly used simple diluents such as water and liquid paraffin, various excipients such as wetting agents, sweeteners, fragrances, and preservatives. Formulations for parenteral administration include sterile aqueous solutions, suspending agents, propylene glycol, polyethylene glycol, vegetable oil such as olive oil, and injectable ester such as ethyl oleate may be used. As a base for suppositories, witepsol, macrogol, tween 61, cacao butter, laurin fat, glycerogelatin, etc. may be used.

The pharmaceutical composition may have any one formulation selected from the group consisting of tablets, pills, powders, granules, capsules, suspensions, oral solutions, emulsions, syrups, sterile aqueous solutions, non-aqueous solutions, suspensions, emulsions, freeze-dried preparations, and suppositories.

In addition, the pharmaceutical composition of the present invention may contain a pharmaceutically effective amount of a RAGE antagonist or a pharmaceutically acceptable salt thereof.

In the present invention, the term “pharmaceutically effective amount” refers to an amount sufficient to treat a disease at a reasonable benefit/risk ratio applicable to any medical treatment. The effective dose level may be determined depending on factors, including the type and severity of the subject's disease, the subject's age and sex, the activity of the drug, the subject's sensitivity to the drug, the time of administration, the route of administration, excretion rate, the period of treatment, and drugs used concurrently, as well as other factors well known in the medical field. The pharmaceutical composition of the present invention may be administered individually or in combination with other therapeutic agents, and may be administered sequentially or simultaneously with conventional therapeutic agents. In addition, the pharmaceutical composition may be administered in a single or multiple dosage form. It is important to administer the pharmaceutical composition in the minimum amount that can exhibit the maximum effect without causing side effects, in view of all the above-described factors, and this amount can be easily determined by a person skilled in the art.

Another aspect provides a method for preventing or treating diabetic stroke, comprising a step of administering a pharmaceutical composition for preventing or treating diabetic stroke, containing at least one of a RAGE (receptor for advanced glycation end-products) antagonist or an AGE (advanced glycation end-products) scavenger, to a subject likely to develop stroke with hyperglycemia or suffering from stroke with hyperglycemia.

The subject likely to develop stroke with hyperglycemia or suffering from stroke with hyperglycemia may be an animal, including or excluding humans.

According to one embodiment, the RAGE antagonist may be one of an AGE antagonist, an HMGB1 antagonist, an S100A4 antagonist, an S100ß antagonist, an S100P antagonist, an amyloid-ß (AB) antagonist, and a RP1 antagonist, without being limited thereto.

In one embodiment, the AGE scavenger may be vitexin, isovitexin, aminoguanidine, thymoquinone, epigallocatechin gallate, rutin, aspirin, or penicillamine.

In one embodiment, the diabetic stroke may be stroke with hyperglycemia. For example, the diabetic stroke may include, but is not limited to, stroke with diabetes, stroke with hyperglycemia, subarachnoid hemorrhage with hyperglycemia, and traumatic brain injury with hyperglycemia.

In one embodiment, the RAGE antagonist may be FPS-ZM1, HMB1 box A, ethyl pyruvate, or azeliragon, without being limited thereto.

The descriptions of the RAGE antagonist, FPS-ZM1, azeliragon, AGE scavenger, HMGB-1, S100A4, S100β, S100P, RP1, prevention, treatment, and diabetic stroke are the same as those described above.

The subject refers to any animals, including humans, that have or are likely to develop diabetic stroke. By administering the pharmaceutical composition of the present invention to a subject suspected of having diabetic stroke, the subject can be treated efficiently.

For example, the subject is a subject in need of prevention or treatment of diabetic stroke. The subject may be, but is not limited to, not only humans but also mammals such as horses, cows, pigs, goats, dogs, cats, camels, rabbits, and sheep, which are in need of treatment of diabetic stroke or similar symptoms.

The administration means introducing the pharmaceutical composition of the present invention to a subject by any appropriate method. The composition may be administered through various routes such as oral or parenteral routes, as long as it can reach the target tissue.

Another aspect of the present invention provides the use of a pharmaceutical composition for preventing or treating diabetic stroke containing the RAGE antagonist and the AGE scavenger.

Another aspect of the present invention provides the use of the RAGE antagonist and the AGE scavenger for preparing a pharmaceutical composition for preventing or treating diabetic stroke.

In the above use, the composition for preventing or treating diabetic stroke, the RAGE antagonist, and the AGE scavenger are as described above.

### Advantageous Effects

Using the composition of the present invention, it is possible to prevent or treat stroke with hyperglycemia. More specifically, the composition of the present invention has the effect of reducing brain injury caused by stroke with hyperglycemia.

### BRIEF DESCRIPTION OF DRAWINGS

FIG. 1A depicts data showing an experimental process over time, and FIGS. 1B and 1C show data measured before and 1 day, 2 days, and 3 days after MCAO for each experimental group. Specifically, FIG. 1B depicts data showing the average value of the measured body weights, and FIG. 1C depicts data showing the average value of scores for the mNSS test.

FIG. 2A depicts data comparing the injured area in the cross-sectional area of the brain between experimental groups, and FIG. 2B depicts data comparing the brain injury volume between experimental groups.

FIG. 3 depicts data showing the protein expression levels of RAGE, AGE, methylglyoxal (MGH1), and GFAP in brain tissue of each experimental group.

FIG. 4 depicts data showing changes in cells by immunohistochemical staining of brain tissue from each of normal rats (sham), a normoglycemic group, and a hyperglycemic group.

FIG. 5 depicts data comparing gene expression levels in brain tissues from normal rats (sham), a normoglycemic group, and a hyperglycemic group.

FIG. 6A depicts data showing an experimental process for a diabetic group over time, and FIGS. 6B and 6C depict data showing the results of measuring blood glucose levels and glycated hemoglobin levels over time in each diabetic group.

FIG. 7A depicts data comparing the injured area in the cross-sectional area of the brain between diabetic groups, and FIG. 7B depicts data comparing the brain injury volume between diabetic groups.

FIG. 8A depicts data showing the expression level of MGH1 by immunohistochemical staining of brain tissue from each diabetic group, and FIG. 8B depicts data comparing the results of quantification. FIG. 8C depicts data showing the level of protein expression in brain tissue, and FIG. 8D depicts data comparing the results of quantification.

FIG. 9 is a standard table that may be used to determine the neurological severity of an individual through each score in a modified neurological severity score (mNSS) test.

### BEST MODE

One aspect provides a pharmaceutical composition for preventing or treating diabetic stroke, containing at least one of a RAGE (receptor for advanced glycation end-products) antagonist or an AGE (advanced glycation end-products) scavenger.

The RAGE is called the receptor for advanced glycation end-products and is a cell membrane multiligand receptor that binds to various types of ligands belonging to the immunoglobulin family. Unlike other multiligand receptors, the various types of ligands that bind thereto are all intracellular products. The advanced glycation end-products (AGEs) result from non-enzymatic glycation and oxidation of proteins, appear in stress-related conditions similar to those in autoimmune connective tissue diseases, and may be formed by oxidation in inflamed tissues or by the myeloperoxidase pathway.

The RAGE antagonist may be a substance that inhibits the action of the RAGE by interfering with the interaction between the RAGE and its ligands.

In one embodiment, the RAGE antagonist may be an AGE antagonist, an HMGB1 antagonist, an S100A4 antagonist, an S100β antagonist, an S100P antagonist, an amyloid-ß (Aß) antagonist, or a RP1 antagonist, without being limited thereto.

In one embodiment, the RAGE antagonist may be FPS-ZM1, HMB1 box A, ethyl pyruvate, or azeliragon, without being limited thereto.

The HMGB-1 (high mobility group box 1), S100A4 (S100 calcium binding protein A4), S100β, S100P (S100 calcium binding protein P), and RP1 (RP1 axonemal microtubule associated) are substances corresponding to ligands for RAGE, and may be substances that promote the action of RAGE by binding to the RAGE. For example, the HMGB-1 antagonist may be HMGB1 box A, or ethyl pyruvate, without being limited thereto.

The RAGE antagonist may be FPS-ZM1. FPS-ZM1 may competitively bind to the domain of RAGE and inhibit the interaction of RAGE with ligands such as AGEs, HMGB1, S100A4, S100β, S100P, amyloid-ß (Aß), and RP1, thereby reducing signaling caused by binding to RAGE. The FPS-ZM1 is a C₂₀H₂₂ClNO compound and refers to a compound represented by Formula 1 below.

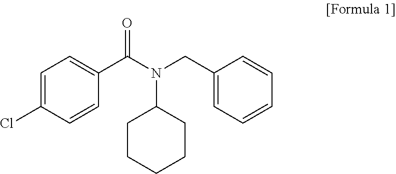 [SMILES: O=C(c1ccc(Cl)cc1)N(Cc1ccccc1)C1CCCCC1]

The RAGE antagonist may be azeliragon. Azeliragon may be referred to as PF-04494700 or TTP488, and refers to a compound represented by Formula 2 below. Azeliragon may be an oral small molecule inhibitor of RAGE.

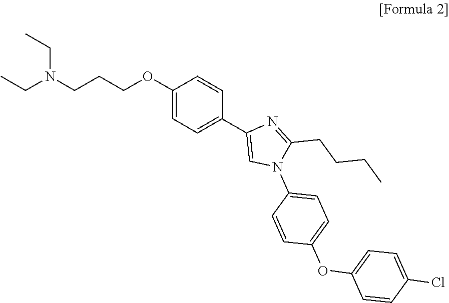 [SMILES: CCCCc1nc(-c2ccc(OCCCN(CC)CC)cc2)cn1-c1ccc(Oc2ccc(Cl)cc2)cc1]

The AGE scavenger may inhibit the formation of AGEs.

In one embodiment, the AGE scavenger may be vitexin, isovitexin, aminoguanidine, thymoquinone, epigallocatechin gallate, rutin, aspirin, or penicillamine.

In one embodiment, the diabetic stroke may be stroke with hyperglycemia. For example, the diabetic stroke may include, but is not limited to, stroke with diabetes, stroke with hyperglycemia, subarachnoid hemorrhage with hyperglycemia, and traumatic brain injury with hyperglycemia.

In one example, diabetic stroke was induced by inducing hyperglycemia in a rat and inducing stroke 34 days later, and hyperglycemic stroke was induced by inducing hyperglycemia in a rat and inducing stroke on day 4. It was confirmed that, in both groups, brain injury was significantly reduced when treated with a RAGE antagonist or an AGE scavenger.

The prevention or preventing refers to any action that inhibits or delays the onset and worsening after onset of diabetic stroke by administering the composition of the present invention.

The treatment or treating refers to any action that alleviates or ameliorates diabetic stroke symptoms and worsening after onset thereof by administering the composition of the present invention.

In one example, it was confirmed that, when stroke occurred in an experimental group with induced hyperglycemia, brain injury in the group significantly increased compared to that in a normoglycemic group. In addition, in one example, it was confirmed that brain injury was significantly reduced when a RAGE antagonist was administered to an experimental group with induced hyperglycemia, and that brain injury was significantly reduced when an AGE scavenger was administered to an experimental group with induced diabetes.

The pharmaceutical composition of the present invention may be used as a single formulation, or it may be prepared and used as a combination formulation further containing a drug known to be effective against stroke with hyperglycemia or against diabetic stroke.

The pharmaceutical composition of the present invention may further contain a pharmaceutically acceptable carrier.

The “pharmaceutically acceptable” means neither significantly stimulating an organism nor inhibiting the biological activity and characteristics of an active material administered.

The carrier may be a natural or non-natural carrier. Depending on the formulation, the composition may be formulated using various carriers such as diluents or excipients, including fillers, extenders, binders, wetting agents, disintegrants, and surfactants. For example, solid formulations for oral administration include tablets, pills, powders, granules, capsules, etc. Such solid formulations are prepared by mixing one or more compounds with at least one excipient, for example, starch, calcium carbonate, sucrose, lactose, gelatin, or the like. In addition to simple excipients, lubricants are also used, such as magnesium stearate, talc, etc. Liquid formulations for oral administration include suspensions, oral solutions, emulsions, and syrups, and may contain, in addition to commonly used simple diluents such as water and liquid paraffin, various excipients such as wetting agents, sweeteners, fragrances, and preservatives. Formulations for parenteral administration include sterile aqueous solutions, suspending agents, propylene glycol, polyethylene glycol, vegetable oil such as olive oil, and injectable ester such as ethyl oleate may be used. As a base for suppositories, witepsol, macrogol, tween 61, cacao butter, laurin fat, glycerogelatin, etc. may be used.

The pharmaceutical composition may have any one formulation selected from the group consisting of tablets, pills, powders, granules, capsules, suspensions, oral solutions, emulsions, syrups, sterile aqueous solutions, non-aqueous solutions, suspensions, emulsions, freeze-dried preparations, and suppositories.

In addition, the pharmaceutical composition of the present invention may contain a pharmaceutically effective amount of a RAGE antagonist or a pharmaceutically acceptable salt thereof.

In the present invention, the term “pharmaceutically effective amount” refers to an amount sufficient to treat a disease at a reasonable benefit/risk ratio applicable to any medical treatment. The effective dose level may be determined depending on factors, including the type and severity of the subject's disease, the subject's age and sex, the activity of the drug, the subject's sensitivity to the drug, the time of administration, the route of administration, excretion rate, the period of treatment, and drugs used concurrently, as well as other factors well known in the medical field. The pharmaceutical composition of the present invention may be administered individually or in combination with other therapeutic agents, and may be administered sequentially or simultaneously with conventional therapeutic agents. In addition, the pharmaceutical composition may be administered in a single or multiple dosage form. It is important to administer the pharmaceutical composition in the minimum amount that can exhibit the maximum effect without causing side effects, in view of all the above-described factors, and this amount can be easily determined by a person skilled in the art.

Another aspect provides a method for preventing or treating diabetic stroke, comprising a step of administering a pharmaceutical composition for preventing or treating diabetic stroke, containing at least one of a RAGE (receptor for advanced glycation end-products) antagonist or an AGE (advanced glycation end-products) scavenger, to a subject likely to develop stroke with hyperglycemia or suffering from stroke with hyperglycemia.

The subject likely to develop stroke with hyperglycemia or suffering from stroke with hyperglycemia may be an animal, including or excluding humans.

According to one embodiment, the RAGE antagonist may be one of an AGE antagonist, an HMGB1 antagonist, an S100A4 antagonist, an S100β antagonist, an S100P antagonist, an amyloid-ß (Aβ) antagonist, and a RP1 antagonist, without being limited thereto.

In one embodiment, the AGE scavenger may be vitexin, isovitexin, aminoguanidine, thymoquinone, epigallocatechin gallate, rutin, aspirin, or penicillamine.

In one embodiment, the diabetic stroke may be stroke with hyperglycemia. For example, the diabetic stroke may include, but is not limited to, stroke with diabetes, stroke with hyperglycemia, subarachnoid hemorrhage with hyperglycemia, and traumatic brain injury with hyperglycemia.

In one embodiment, the RAGE antagonist may be FPS-ZM1, HMB1 box A, ethyl pyruvate, or azeliragon, without being limited thereto.

The descriptions of the RAGE antagonist, FPS-ZM1, azeliragon, AGE scavenger, HMGB-1, S100A4, S100β, S100P, RP1, prevention, treatment, and diabetic stroke are the same as those described above.

The subject refers to any animals, including humans, that have or are likely to develop diabetic stroke. By administering the pharmaceutical composition of the present invention to a subject suspected of having diabetic stroke, the subject can be treated efficiently.

For example, the subject is a subject in need of prevention or treatment of diabetic stroke. The subject may be, but is not limited to, not only humans but also mammals such as horses, cows, pigs, goats, dogs, cats, camels, rabbits, and sheep, which are in need of treatment of diabetic stroke or similar symptoms.

The administration means introducing the pharmaceutical composition of the present invention to a subject by any appropriate method. The composition may be administered through various routes such as oral or parenteral routes, as long as it can reach the target tissue.

### MODE FOR INVENTION

Hereinafter, one or more embodiments will be described in more detail by way of examples. However, these examples are intended to illustrate one or more embodiments and the scope of the present invention is not limited to these examples.

### Example 1: Evaluation of Brain Injury Following FPS-ZM1 Treatment

### 1-1. Experimental Method

For the experiment, experimental animals were classified as shown in Table 1 below and tested.

TABLE 1

| Experimental group | Method |
|---|---|
| Hyperglycemic group | Rats administered |
| | streptozotocin (STZ) and |
| | subjected to 30 minutes of |
| | tMCAO |
| Hyperglycemic group | Rats administered |
| treated with FPS-ZM1 | streptozotocin (STZ) and |
| | subjected to 30 minutes of |
| | tMCAO, followed by treatment |
| | with FPS-ZM1 |
| Normoglycemic group | Rats subjected to 2 hours of |
| | tMCAO |
| Normoglycemic group | Rats subjected to 2 hours of |
| treated with FPS-ZM1 | tMCAO and then treated with |
| | FPS-ZM1 |

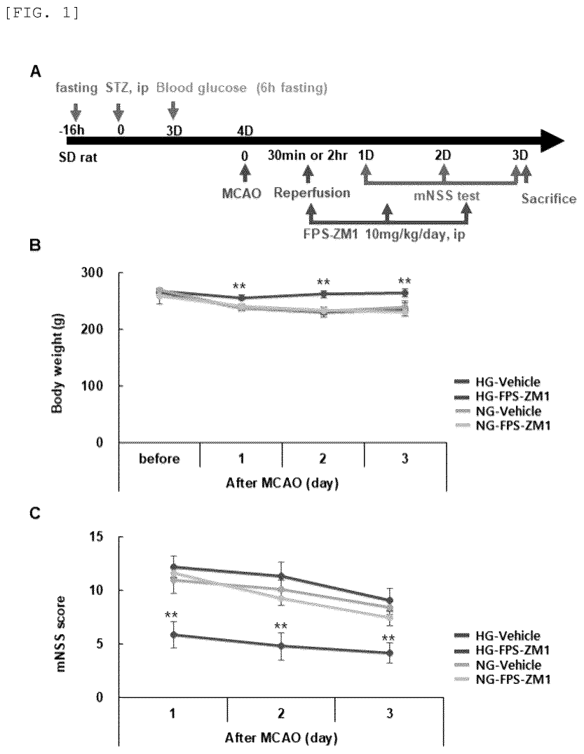

Experimental animal model production and the experiment were carried out as shown in FIG. 1A. Specifically, these were carried out as follows. To produce an experimental animal model, 7-week-old male SD rats were fasted for 16 hours. Thereafter, streptozotocin (STZ) was administered to the rats, and after 3 days, fasting blood glucose levels were measured in the tail vein, and only animals with a blood glucose level of 300 mg/dl or more were used as a hyperglycemic model. A cerebral ischemia model was produced by subjecting the produced hyperglycemic model or a normoglycemic model to temporary middle cerebral artery occlusion (tMCAO). For MCAO, a coated suture was inserted through the right external carotid artery into the internal carotid artery, and inserted approximately 19 mm up to the middle cerebral artery (MCA). 30 minutes or 2 hours after insertion, the coated suture was removed, and a reperfusion model was used as a cerebral ischemia model. Since cerebral infarction was rarely found in normoglycemic animals after 30-minute MCAO, a model subjected to 2 hours of MCAO was used and compared with the hyperglycemic group. The normoglycemic group (NG), after 2 hours of MCAO, was divided into a vehicle group and an FPS-ZM1 group and used in the experiment. The hyperglycemic group (HG), after 30 minutes of MCAO, was divided into a vehicle group and an FPS-ZM1 group and used in the experiment. The FPS-ZM1 group in each of the normoglycemic group and the hyperglycemic group was injected intraperitoneally with 10 mg/kg of FPS-ZM1 within 5 minutes after MCAO reperfusion and injected with FPS-ZM1 once a day. After surgery, the mNSS (modified neurological severity score) test shown in FIG. 9 and weight measurement were conducted at a certain time every day to check body weight changes and neurobehavioral deficits of the experimental animals. In addition, rats were sacrificed 3 days after MCAO, and plasma and brain tissue were collected and analyzed.

1-2. Body Weight Changes and mNSS Test Results

After performing MCAO, body weight measurement and the mNSS test were performed at the same time every day. As a result, as shown in FIG. 1B, it was confirmed that, regarding body weight changes, there was no reduction in body weight only in the hyperglycemic group administered FPS-ZM1 (hereinafter referred to as “HG-FPS-ZM1”), and the body weight in the remaining groups decreased compared to the initial body weight before performing MCAO.

In addition, as shown in FIG. 1C, it was confirmed that the mNSS score in the HG-FPS-ZM1 group was significantly lower than those in the other groups, indicating that neurobehavioral deficits in the HG-FPS-ZM1 group were very small compared to those in the other groups subjected to MCAO.

### 1-3. Results of Evaluation of Brain Injury

To evaluate brain injury, the volume of cerebral infarction was determined by staining the entire brain with cresyl violet at 1-mm intervals.

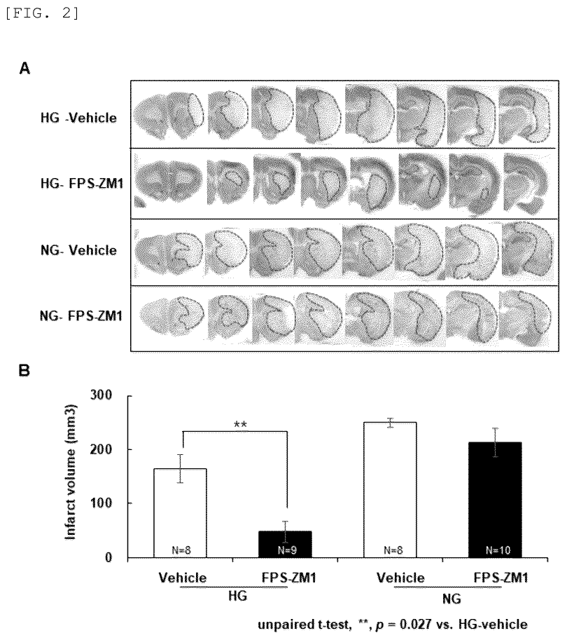

As shown in FIG. 2A, as a result of comparing the HG-vehicle group, subjected to 30 minutes of MCAO, with the HG-FPS-ZM1 group administered FPS-ZM1 after MCAO, which were hyperglycemic experimental animals, it was confirmed that the cross-sectional area of injury significantly decreased in the FPS-ZM1-administered group, and that the volume of actual injury was also more significant in the HG-FPS-ZM1 group than in the HG-vehicle group.

On the other hand, as shown in FIG. 2B, in the normoglycemic group, it was difficult through the area photographs to confirm the degree of reduction in the cross-sectional area of injury in the NG-FPS-ZM1 group administered FPS-ZM1 compared to the NG-vehicle group, and it was confirmed that the overall injury volume also decreased slightly in the NG-FPS-ZM1 group, but this decrease was not significant.

### 1-4. Analysis of Protein Expression by Western Blot Analysis

After all experiments were completed, the protein expression levels of RAGE, AGE, and methylglyoxal (MGH1) in the brain tissue from each experimental group were analyzed by Western blot analysis. For comparison, protein expression in the brain tissue from normal rats (sham) was used as a control.

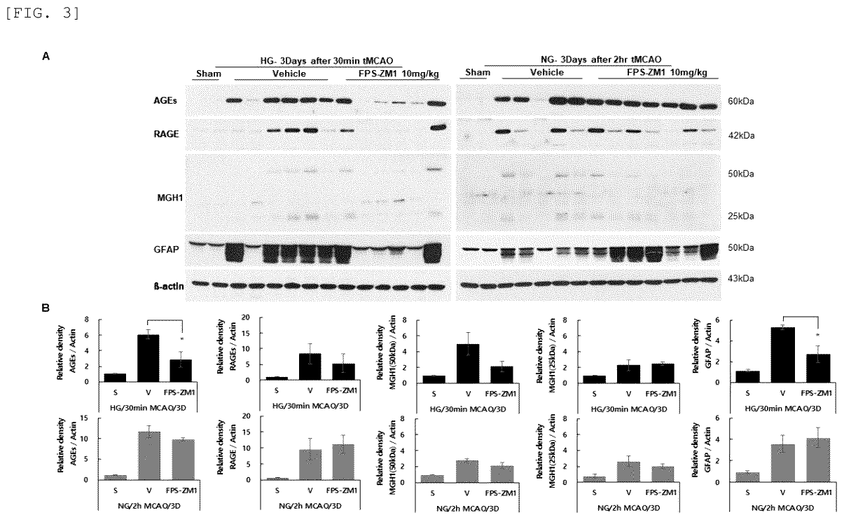

As a result, as shown in FIG. 3, it was confirmed that there was no significant difference in MGH1 between all the groups, and the expression levels of AGE, RAGE, and GFAP in both the NG-vehicle group and the HG-vehicle group after MCAO were significantly higher than those in the sham group. In addition, it was confirmed that the expression levels of AGE and GFAP were significantly lower in the HG-FPS-ZM1 group than in the HG-vehicle group.

In addition, it was confirmed that, in the normoglycemic group, there was no significant difference in the expression levels of RAGE and AGE between the FPS-ZM1-administered group (NG-FPS-ZM1) and the non-administered group (NG-vehicle).

### 1-5. Analysis of Changes in Proteins in Cells by Immunohistochemical Staining

The degree of neuronal cell death in the cerebral cortex from each experimental group was analyzed using cresyl violet staining. In addition, immunohistochemistry was used to analyze the level of activity of astrocytes and microglia and the expression levels of AGE and MGH1 in the cells.

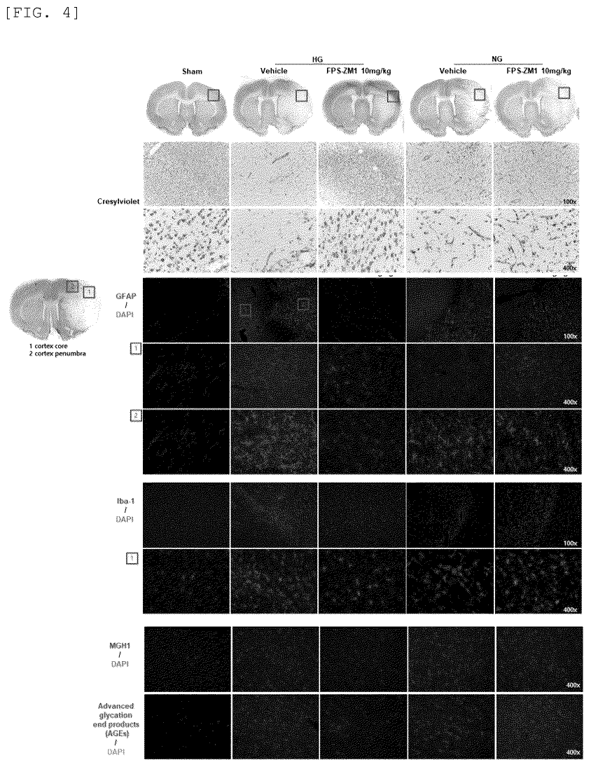

As a result, as shown in FIG. 4, it was observed in cresyl violet staining that the degree of neuronal cell death was higher in all of the HG-vehicle group, NG-vehicle group, and NG-FPS-ZM1 group after MCAO than in the sham group, and the number of live cells was larger in the HG-FPS-ZM1 group. GFAP (glial fibrillary acidic protein) staining showed that activated astrocytes increased and were less observed in the HG-FPS-ZM1 group. This was also observed in microglial cells through Iba-1 staining. Even in intracellular AGE and MGH1 staining after MCAO, activated astrocytes were less observed in the HG-FPS-ZM1 group than in the sham group.

### 1-6. Analysis of Gene Expression by Real-Time Polymerase Chain Reaction

The expression levels of the following vascular-related factors in brain tissue from each experimental group were analyzed using real-time polymerase chain reaction (real-time PCR): ZO-1, occludin, and claudin, which are involved in tight junctions; Tie-2, a weakening factor (recovery factor) that increases blood brain barrier permeability; VEGF, Ang-1, and MMP-2, which are angiogenic factors involved in the migration and proliferation of endothelial cells; and MMP-9, which is a representative substance implicated in basement membrane destruction and also plays an important role in inflammatory diseases.

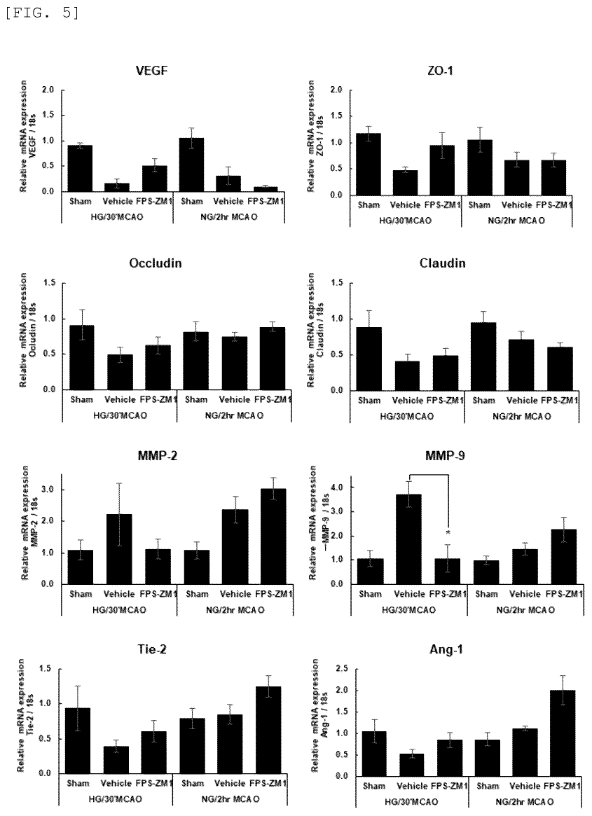

As a result, as shown in FIG. 5, it was confirmed that the expression levels of most vascular-related factors decreased in the HG-vehicle and NG-vehicle groups after MCAO compared to the sham group, and increased in the HG-FPS-ZM1 group compared to the NG-FPS-ZM1 group, but there was no significant difference between the groups. On the other hand, it was confirmed that the expression level of MMP-9 significantly increased in the HG-vehicle group compared to the sham group, and significantly decreased in the HG-FPS-ZM1 group to a level similar to that in the sham group.

### Example 2: Evaluation of Brain Injury Following Aminoguanidine Treatment

### 2-1. Experimental Method

For the experiment, experimental animals were classified as shown in Table 2 below and tested.

TABLE 2

| Experimental group | Method |
|---|---|
| Diabetic group (vehicle) | Rats administered |
| | streptozotocin (STZ) and then |
| | subjected to 30 minutes of |
| | tMCAO on day 34 |
| Diabetic group treated | Rats administered |
| with aminoguanidine (AG) | streptozotocin (STZ), treated |
| | with aminoguanidine starting |
| | from day 4 after administration, |
| | and subjected to 30 minutes of |
| | tMCAO on day 34 |

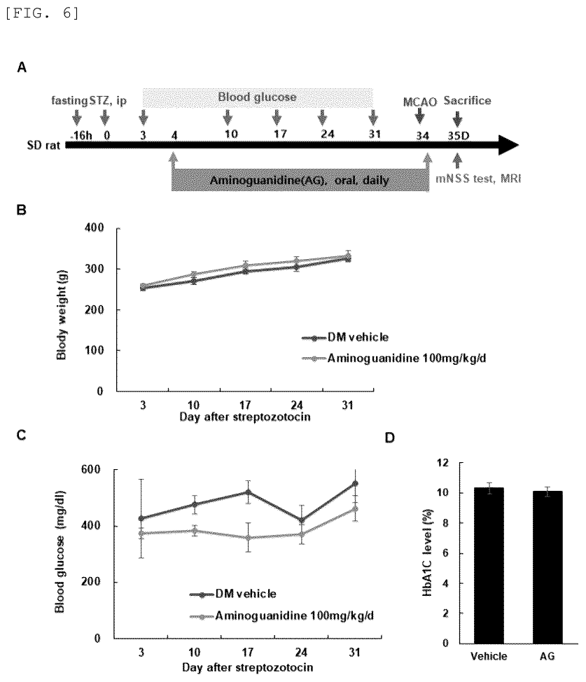

Experimental animal model production and the experiment were carried out as shown in FIG. 6A. Specifically, these were carried out as follows. To produce an experimental animal model of diabetes mellitus (DM), 7-week-old male SD rats were fasted for 16 hours. Thereafter, streptozotocin (STZ) was administered to the rats, and on day 3, blood glucose levels were measured. The rats were divided into a vehicle group and an aminoguanidine (AG) group, which had similar average blood glucose levels, and were maintained for 34 days. The AG group was orally administered 100 mg/kg of aminoguanidine once a day starting from day 4 after diabetes induction, and was finally administered aminoguanidine within 30 minutes after MCAO. On day 1 after MCAO, the mNSS test shown in FIG. 9 was performed, and then cerebral infarction was checked using an animal MRI system. Next, the rats were sacrificed, and plasma and brain tissue were collected and analyzed. For comparison, protein expression in the brain tissue from normal rats (sham) was used as a control.

### 2-2. Checking of Body Weight Changes, Blood Glucose Level Changes, and Glycated Hemoglobin Levels

The impact on diabetes was evaluated by checking changes in body weight and blood glucose levels once a week after diabetes induction and checking final glycated hemoglobin levels after MCAO.

As a result, as shown in FIG. 6B, it was confirmed that the change in body weight was not significantly different between the DM-vehicle group and the AG (aminoguanidine) group. In addition, as shown in FIGS. 6C and 6D, there was no significant difference in blood glucose level and glycated hemoglobin level between the two groups.

### 2-3. Results of Evaluation of Brain Injury

To evaluate brain injury, the volume of cerebral infarction was determined using an animal MRI system.

As a result, as shown in FIG. 7, it was confirmed that there was no significant difference in brain injury between the DM-vehicle group obtained by subjecting diabetic animals to 30 minutes of MCAO and the group administered AG starting from day 4 after diabetes induction.

### 2-4. Analysis of Protein Expression by Immunohistochemical Staining and Western Blot Analysis

Day 1 after MCAO, the expression levels of MGH1 in the brain tissues from the diabetic groups were analyzed by immunohistochemical staining and Western blot analysis.

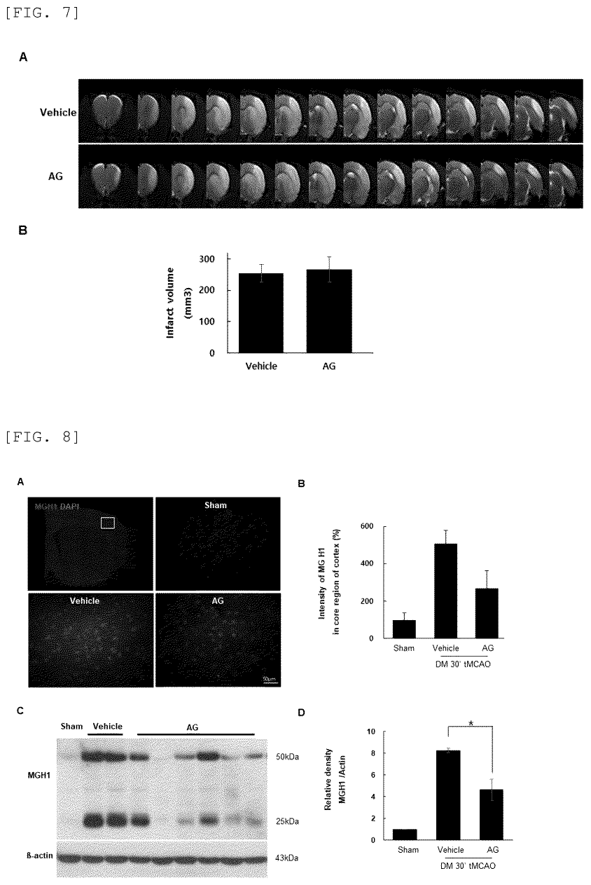

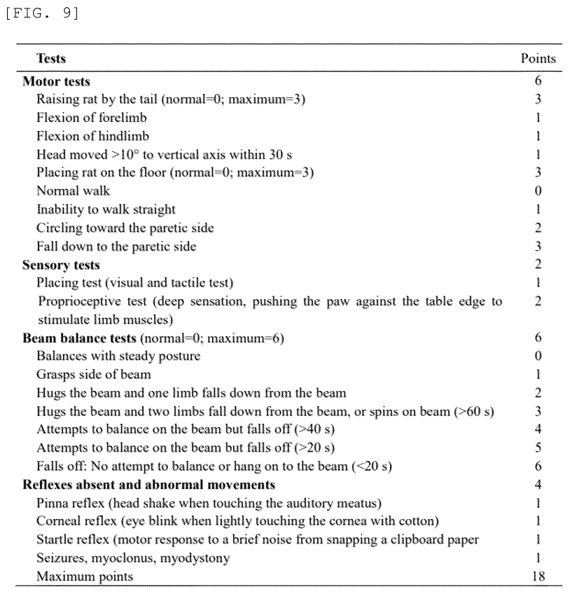

As a result, as shown in FIG. 8, it was confirmed that the expression level of MGH1 significantly increased in the vehicle group compared to the sham group, and decreased in the AG group.

So far, the present invention has been described with reference to the embodiments. Those of ordinary skill in the art to which the present invention pertains will appreciate that the present invention may be embodied in modified forms without departing from the essential characteristics of the present invention. Therefore, the disclosed embodiments should be considered from an illustrative point of view, not from a restrictive point of view. The scope of the present invention is defined by the claims rather than the foregoing description, and all differences within the scope equivalent thereto should be construed as being included in the present invention.

### INDUSTRIAL APPLICABILITY

Using the composition of the present invention, it is possible to prevent or treat stroke with hyperglycemia. More specifically, the composition of the present invention has the effect of reducing brain injury caused by stroke with hyperglycemia.

1. A method for preventing or treating a subject having diabetic stroke, comprising administering a pharmaceutical composition comprising at least one of a RAGE (receptor for advanced glycation end-products) antagonist or an AGE (advanced glycation end-products) scavenger.

2. The method of claim 1, wherein the RAGE antagonist is at least one of an AGE antagonist, an HMGB1 antagonist, an S100A4 antagonist, an S100ß antagonist, an S100P antagonist, an amyloid-ß (Aß) antagonist, or a RP1 antagonist.

3. The method of claim 1, wherein the AGE scavenger is at least one of vitexin, isovitexin, aminoguanidine, thymoquinone, epigallocatechin gallate, rutin, aspirin, or penicillamine.

4. The method of claim 1, wherein the diabetic stroke is stroke with hyperglycemia.

5. The method of claim 1, wherein the subject is likely to develop stroke with hyperglycemia or the subject is suffering from stroke with hyperglycemia.
